(19) United States
(12) Patent Application Publication
(10) Pub. No.: US 2013/0079425 A1
(43) Pub. Date: Mar. 28, 2013

(54) Urokinase-Type Plasminogen Activator Protein /Plasminogen Activator Inhibitor Type-1 Protein Selected Reaction Monitoring Assay

(71) Applicant: Expression Pathology, Inc., Rockville, MD (US)

(72) Inventor: David B. KRIZMAN, Gaithersburg, MD (US)

(73) Assignee: EXPRESSION PATHOLOGY, INC., Rockville, MD (US)

(21) Appl. No.: 13/685,446

(22) Filed: Nov. 26, 2012

Related U.S. Application Data
(63) Continuation of application No. PCT/US2011/038196, filed on May 26, 2011.
(60) Provisional application No. 61/348,712, filed on May 26, 2010.

(51) Int. Cl. C12Q 1/37

(52) U.S. Cl. CPC C12Q 1/37
USPC 514/789; 435/24

(57) ABSTRACT

Specific peptides are provided that are derived from subsequences of the urokinase-type plasminogen activator protein and the plasminogen activator inhibitor type-1 protein along with assays that can measure those peptides directly in complex protein lysate samples, including protein lysates prepared from histologicaly-processed formalin fixed tissue. The presence and amount of those peptides in samples from a subject can be associated with disease, including cancer, in a subject and provide information about the diagnostic stage/grade/status of the disease/cancer.

This application claims the benefit of U.S. Provisional Application No. 61/348,712, filed May 26, 2010, entitled “Urokinase-Type Plasminogen Activator Protein/Plasminogen Activator Inhibitor Type-1 Protein-Selected Reaction Monitoring Assay” naming as an inventor David B. Krizman, the entirety of which is incorpoated by reference.

### INTRODUCTION

Specific peptides are provided that are derived from subsequences of the urokinase-type plasminogen activator protein, which will be referred to as uPA, and from subsequences of the plasminogen activator inhibitor type-1 protein, which will be referred to as PAI-1. Specific characteristics about each peptide are provided, which includes the peptide sequence and fragmentation/transition ions for reliable, accurate and consistent analysis in mass spectrometric analysis. Also described is the use of those peptides in a mass spectrometry-based Selected Reaction Monitoring (SRM), which can also be referred to as a Multiple Reaction Monitoring (MRM) assay. This SRM assay can be used to measure relative or absolute quantitative levels of one or more of the specific peptides from the uPA and PAI-1 proteins and therefore provide a means of measuring the amount of the uPA and PAI-1 proteins by mass spectrometry in a given protein preparation obtained from a biological sample.

More specifically, the SRM assay can measure these peptides directly in complex protein lysate samples prepared from cells procured from patient tissue samples, such as formalin fixed cancer patient tissue. Methods of preparing protein samples from formalin-fixed tissue are described in U.S. Pat. No. 7,473,532, the contents of which are hereby incorporated by references in their entirety. The methods described in U.S. Pat. No. 7,473,532 may conveniently be carried out using Liquid Tissue® reagents available from Expression Pathology, Inc. (Rockville, Md.).

Results from the SRM assay can be used to correlate accurate and precise quantitative levels of the uPA and PAI-1 proteins with the specific cancer of the patient from whom the tissue was collected. This not only provides diagnostic information about the cancer, but also permits a physician or other medical professional to determine appropriate therapy for the patient. Such an assay that provides diagnostically important information about levels of protein expression in a diseased tissue or other patient sample is termed a companion diagnostic assay. For example, such an assay can be designed to diagnose the stage or degree of a cancer and determine which therapeutic agent, or course of therapy, to which a patient is most likely to respond with a positive outcome.

The assays described herein measure relative or absolute levels of specific unmodified peptides from the uPA and PAI-1 proteins and also can measure absolute or relative levels of specific modified peptides from the uPA and PAI-1 proteins. Examples of modifications include phosphorylated amino acid residues and glycosylated amino acid residues that are present on the peptides.

Relative quantitative levels of the uPA and PAI-1 proteins are determined by the SRM methodology whereby the chromatographic peak area (or the peak height if the peaks are sufficiently resolved) of an individual peptide, or multiple peptides, from the uPA and PAI-1 proteins in one biological sample is compared to the chromatographic peak area determined for the same identical uPA and PAI-1 peptides, or peptides, using the same methodology in one or more additional and different biological samples. In this way, the amount of a particular peptide, or peptides, from the uPA and PAI-1 proteins, and therefore the amount of the uPA and PAI-1 proteins, is determined relative to the same uPA and PAI-1 peptide, or peptides, across 2 or more biological samples under the same experimental conditions. In addition, relative quantitation can be determined for a given peptide, or peptides, from the uPA and PAI-1 proteins within a single sample by comparing the chromatographic peak area for that peptide by SRM methodology to the chromatographic peak area for another and different peptide, or peptides, from a different protein, or proteins, within the same protein preparation from the biological sample. In this way, the amount of a particular peptide from the uPA and PAI-1 proteins, and therefore the amount of the uPA and PAI-1 proteins, is determined relative one to another within the same sample. These approaches generate quantitation of an individual peptide, or peptides, from the uPA and PAI-1 proteins to the amount of another peptide, or peptides, between samples and within samples wherein the amounts as determined by chromatographic peak area are relative one to another, regardless of the absolute weight to volume or weight to weight amounts of the uPA and PAI-1 peptides in the protein preparation from the biological sample. Relative quantitative data about individual chromatographic peak areas between different samples are normalized to the amount of protein analyzed per sample. Relative quantitation can be performed across many peptides simultaneously in a single sample and/or across many samples to gain insight into relative protein amounts, one peptide/protein with respect to other peptides/proteins.

Absolute quantitative levels of the uPA and PAI proteins are determined by the SRM methodology whereby the chromatographic peak area of an individual peptide from the uPA and PAI-1 proteins in one biological sample is compared to the chromatographic peak area of a spiked internal standard, where the internal standard is a synthetic version of the same exact uPA and PAI-1 peptides that contains one or more amino acid residues labeled with one or more heavy isotopes. The internal standard is synthesized so that when analyzed by mass spectrometry it generates a predictable and consistent signature chromatographic peak that is different and distinct from the native uPA and PAI-1 peptide chromatographic signature peak. Thus when the internal standard is spiked into a protein preparation from a biological sample in known amounts and analyzed by mass spectrometry, the signature chromatographic peak area of the native peptide is compared to the signature chromatographic peak area of the internal standard peptide, and this numerical comparison indicates either the absolute molarity or absolute weight of the native peptide present in the original protein preparation from the biological sample. Absolute quantitative data for fragment peptides are displayed according to the amount of protein analyzed per sample. Absolute quantitation can be performed across many peptides, and thus proteins, simultaneously in a single sample and/or across many samples to gain insight into absolute protein amounts in individual biological samples and in cohorts of individual samples.

The assay methods can be used to aid diagnosis of the stage of cancer, for example, directly in patient-derived tissue, such as formalin fixed tissue, and to aid in determining which therapeutic agent, and which therapeutic strategy, would be most advantageous for use in treating that patient. Cancer tissue that is removed from a patient either through surgery, such as for therapeutic removal of partial or entire tumors, or through biopsy procedures conducted to determine the presence or absence of suspected disease, is analyzed to determine whether or not a specific protein, or proteins, and which forms of proteins, are present in that patient tissue. Moreover, the expression level of the protein(s) can be determined and compared to a “normal” or reference level found in healthy tissue or tissue that shows a different stage/grade of cancer. This information can then be used to assign a stage or grade to a specific cancer and can be matched to a strategy for treating the patient based on the determined levels of specific proteins. Matching specific information about levels of the uPA and PAI-1 proteins, as determined by an SRM assay, to a treatment strategy that is based on levels of these proteins in cancer cells derived from the patient defines what has been termed a personalized medicine approach to treating disease. The assay methods described herein form the foundation of a personalized medicine approach by using analysis of proteins from the patient's own tissue as a source for diagnostic and treatment decisions.

Advantageously, both of these proteins, uPA and PAI-1, have been demonstrated in clinical studies and reported in the scientific literature to be highly useful for predicting the likely course of disease for some cancers, including breast cancer. Increased levels of either protein in patient-derived frozen cancer tissue, as assayed by the ELISA method, indicates tissue that the tumor tissue contains aggressively growing cells and consequently associate with a less favorable outcome for that patient. In addition, increasing levels of either protein as demonstrated in frozen patient tissue is also associated with the need to treat the patient with adjuvant chemotherapy, treating specifically with the CMF treatment regimen. There is strong scientific literature that associates measured levels of the proteins with outcome forecast and treatment decisions. The current ELISA method that is used to quantitatively measure these proteins provides such information only in patient tissue that has been preserved by freezing and storage at −80° C. However, this assay does not provide comparable information in formalin fixed patient tissue and thus an assay that can provide such quantitative measurement of these proteins directly in formalin fixed tissue would he highly advantageous. This is because the overwhelming majority of patient tissue is preserved by fixation in formalin, not by freezing and storing at −80° C.

### DETAILED DESCRIPTION

In principle, any predicted peptide derived from the uPA and PAI-1 proteins, for example by digesting with a protease of known specificity (e.g. trypsin), can be used as a surrogate reporter to determine the abundance of uPA and PAI-1 proteins in a sample using a mass spectrometry-based SRM assay. Similarly, any predicted peptide sequence containing an amino acid residue at a site that is known to be potentially modified in the uPA and PAI-1 proteins also might potentially be used to assay the extent of modification of the uPA and PAI-1 proteins in a sample. Surprisingly, however, the present inventors have found that many potential peptide sequences are unsuitable or ineffective for use in mass spectrometry-based SRM assays. The peptides might, for example, be difficult to detect by mass spectrometry, or may be unstable to the conditions used to obtain the peptides from the parent protein. This is especially found to be the case when interrogating protein lysates prepared from formalin fixed tissue using the Liquid Tissue® protocol provided in U.S. Pat. No. 7,473,532. Unexpectedly it was found to be advantageous to experimentally identify preferred modified and unmodified peptides in actual Liquid Tissue® lysates in order to develop a reliable and accurate SRM assay for the uPA and PAI-1 proteins. Preferred modified and unmodified peptides for use in the mass spectrometric methods described herein (e.g., SRM), including identifying presence (or absence) and/or amount of proteins in formalin fixed tissues, are hereinafter known as optimized peptides.

In general, peptides were derived from the uPA and PAI-1 proteins in the course of the protease digestion of the proteins within a complex Liquid Tissue® lysate prepared from cells procured from formalin fixed patient tissue. The Liquid Tissue® lysates were then analyzed by mass spectrometry to determine those peptides derived from uPA and PAI-1 proteins that are preferably detected and analyzed by mass spectrometry (i.e., optimized preferred modified and unmodified peptides). The results are employed to identify a specific subset of preferred peptides selected for their suitability in mass spectrometric analysis. The procedure employed permits experimental determination of peptides or peptides fragments that ionize most effectively, and which provide suitable data for resulting peptide transition fragment ions that can be identified and quantitated in a Liquid Tissue® preparation from formalin fixed patient tissue. These results can then be compared to results obtained by mass spectrometry analysis of the recombinant protein that has been digested with the same protease, or proteases, in order to confirm the existence of preferred or optimized peptides and their resulting transition fragments.

In addition to their suitability in mass spectrometric analysis, the ability of labeled versions of preferred (or more specifically optimized) peptides to withstand the conditions used in Liquid Tissue® preparation protocols is an important determinant as to which peptides are preferred (or optimized where formalin fixed tissue is used) for qualitative or quantitative analyzing of tissues by mass spectrometry (e.g., SRM). This latter property depends not only on the amino acid sequence of the peptide but also on the ability of a modified residue within a peptide to survive in modified form during the sample preparation. The assay method described below can be used to identify the peptides from uPA and PAI-1 proteins that are preferred or optimized for identifying and quantitating protein expression or modification in patient samples, and more specifically patient samples derived from formalin fixed tissue, by mass spectrometry-based SRM assay.

Assay Method for the Identification of Peptides from uPA and/or PAI-1
1. Identification of a preferred (or optimized) fragment peptide, or preferred (or optimized) fragment peptides, for both the uPA and PAI-1 proteins
   a. Treat purified uPA protein with the Liquid Tissue® reagents and protocol using a protease or proteases, (that may or may not include trypsin), to digest the uPA protein. Analyze some (e.g., 10, 20, 30 or 40%), most (e.g., more than 50, 60, 70, 80, 90, 95, 98 or 99%), or all resulting protein fragments by tandem mass spectrometry and identify all fragment peptides from the uPA protein, where individual fragment peptides do not contain any peptide modifications such as phosphorylations or glycosylations.
   b. Treat purified PAI-1 protein with the Liquid Tissue® reagents and protocol using a protease or proteases, (that may or may not include trypsin), to digest the PAI-1 protein. Analyze some, most, or all resulting protein fragments by tandem mass spectrometry and identify some, most, or all fragment peptides from the PAI-1 protein, where individual fragment peptides do not contain any peptide modifications such as phosphorylations or glycosylations.
   c. Prepare a Liquid Tissue® protein lysate from a formalin fixed biological sample using the same protease or proteases as utilized when preparing the purified uPA protein (that may or may not include trypsin), to digest most, or all proteins. Analyze some, most, or all resulting protein fragments from some, most, or all proteins in the mixture by tandem mass spectrometry and identify some, most, or all fragment peptides specifically from the uPA protein, where individual fragment peptides do not contain any peptide modifications such as phosphorylations or glycosylations. Analyze some, most, or all resulting protein fragments from some, most, or all the proteins by tandem mass spectrometry and identify some, most, or all fragment peptides from the uPA protein that carry peptide modifications such as for example phosphorylated or glycosylated residues.
   d. Prepare a Liquid Tissue® protein lysate from a formalin fixed biological sample using the same protease or proteases as utilized when preparing the purified PAI-1 protein (that may or may not include trypsin), to digest most, or all proteins. Analyze some, most, or all resulting protein fragments from some, most, or all proteins by tandem mass spectrometry and identify some, most, or all fragment peptides from the PAI-1 protein, where individual fragment peptides do not contain any peptide modifications such as phosphorylations or glycosylations. Analyze some, most, or all protein fragments by tandem mass spectrometry and identify some, most, or all fragment peptides from the PAI-1 protein that carry peptide modifications such as for example phosphorylated or glycosylated residues.
   e. Some, most or all peptides generated by a specific digestion method from the entire, full length uPA and PAI-1 proteins potentially can be measured, but preferred peptides are those that are identified by mass spectrometry from analysis of the purified proteins and that also are identified directly in a complex Liquid Tissue®protein lysate prepared from a histopathologically fixed biological sample (e.g., optimized peptides can be measured where the tissue is formalin fixed). Peptides that are post-translationally modified, and their specific fragment characteristics, can be considered preferred or optimized peptides and assayed where the relative levels of the modified peptides are determined in the same manner as determining relative amounts of unmodified peptides for the uPA and PAI-1 proteins.
2. Mass Spectrometry Assay for Fragment Peptides from the uPA and PAI-1 Proteins
   a. A Selected Reaction Monitoring (SRM), or also known as a Multiple Reaction Monitoring (MRM), assay is conducted on a triple quadrupole mass spectrometer for each individual preferred or optimized fragment peptides identified in a Liquid Tissue® lysate from the uPA and PAI-1 proteins may be developed as follows:
      i. Determine retention time for each fragment peptide of uPA or PAI-1 for at least one suitable fractioning method including but not limited to gel electrophoresis, liquid chromatography, capillary electrophoresis, isoelectric separation chromatography, nano-reversed phase liquid chromatography, high performance liquid chromatography, or reverse phase high performance liquid chromatography.
      ii. Identify suitable fragment transition ions to monitor for one or more fragment peptides based on the highest signal to noise ratio and/or the lowest standard deviation between replicate analyses for use in Liquid Tissue® samples prepared from histopathologically fixed tissue (e.g., formalin fixed tissues).
   b. Perform SRM/MRM analysis so that the amount of the fragment peptide, or peptides, of the uPA and PAI-1 proteins that is detected, as a function of specific peak area (or height where suitable) from an SRM/MRM mass spectrometry analysis, reflects both the relative and absolute amount of the protein in a particular Liquid Tissue® lysate.
      i. Relative quantitation is achieved by: comparing the (e.g., electrophoretic chromatographic, etc.) peak area for a fragment peptide to the peak area of the same fragment peptide, or other fragment peptides from other proteins, in other samples derived from different and separate biological sources, where the chromatographic peak area comparison between the samples for a peptide fragment are normalized to amount of protein analyzed in each sample. Comparison of the separation peak area for a given fragment peptide to the separation peak areas from other fragment peptides derived from different proteins within the same sample can be performed to normalize changing levels of one protein to levels of other proteins that do not change their levels of expression under various conditions (e.g., cellular conditions). Relative quantitation can be applied to unmodified fragment peptides and modified fragment peptides, where the modifications include but are not limited to phosphorylation and/or glycosylation, and where the relative levels of modified peptides are determined in the same manner as determining relative amounts of unmodified peptides.
      ii. Absolute quantitation of a given peptide is achieved by comparing the peak area for a given fragment peptide in an individual biological sample to the peak area of an internal fragment peptide standard spiked into the protein lysate from the biological sample. The analysis of the given peptide and the standard spiked into the sample can be conducted simultaneously. The internal standard can be a labeled version (e.g., a labeled synthetic version) consisting of the exact amino acid sequence of the fragment peptide that is being interrogated. The labeled standard is spiked into a sample in known amounts, and the chromatographic peak area can be determined for both the internal fragment peptide standard and the native fragment peptide in the biological sample separately, followed by comparison of both peak areas and deriving the absolute amount of the native peptide as compared to the absolute amount of the spiked peptide standard.
         Absolute quantitation can be applied to unmodified fragment peptides and modified fragment peptides, where the modifications include but are not limited to phosphorylation and/or glycosylation, and where the absolute levels of modified peptides can be determined in the same manner as determining absolute levels of unmodified peptides.
3. Associate Fragment Peptide Quantitation to Cancer Diagnosis and/or Treatment
   a. Perform relative and/or absolute quantitation of fragment peptide levels of the uPA and PAI-1 proteins and associate results with the stage/grade/status of cancer in patient tumor tissue; and/or
   b. Perform relative and/or absolute quantitation of fragment peptide levels of the uPA and PAI-1 proteins and correlate with specific and different treatment strategies, wherein this correlation has been, or can be demonstrated in the future, to correlate with outcome to various treatment strategies through correlation studies across cohorts of patients and tissue from those patients. Once either previously established correlations are confirmed by this assay, or new correlations established, the assay method can be used to associate quantitative results for the uPA and PAI-1 proteins in patient tissue to more effective patient treatment strategy.

TABLE 1
uPA Peptide Sequences and Specific Characteristics

| | | Mono- | Precursor | | Product | |
|---|---|---|---|---|---|---|
| | | isotopic | charge | Precursor | Transition | Ion |
| SEQ ID | Peptide sequence | Mass | state | m/z | m/z | Type |
| SEQ ID NO: 1 | KPSSPPEELK | 1111.59937 | 2 | 556.30 | 389.24 | y3 |
| | | | 2 | 556.30 | 518.28 | y4 |
| | | | 2 | 556.30 | 615.33 | Y5 |
| | | | 2 | 556.30 | 712.39 | y6 |
| | | | 2 | 556.30 | 799.42 | y7 |
| | | | 2 | 556.30 | 886.45 | y8 |
| | | | 2 | 556.30 | 983.50 | y9 |
| | | | 2 | 556.30 | 1111.60 | y10 |
| SEQ ID NO: 2 | DYSADTLAHHNDIALLK | 1896.94502 | 2 | 948.98 | 373.28 | y3 |
| | | | 2 | 948.98 | 444.32 | y4 |
| | | | 2 | 948.98 | 557.40 | y5 |
| | | | 2 | 948.98 | 672.43 | y6 |
| | | | 2 | 948.98 | 786.47 | y7 |
| | | | 2 | 948.98 | 923.53 | y8 |
| | | | 2 | 948.98 | 1060.59 | y9 |
| | | | 2 | 948.98 | 1131.63 | y10 |
| | | | 2 | 948.98 | 1244.71 | y11 |
| | | | 2 | 948.98 | 1345.76 | y12 |
| | | | 2 | 948.98 | 1460.79 | y13 |
| | | | 3 | 632.99 | 672.43 | y6 |
| | | | 3 | 632.99 | 786.47 | y7 |
| | | | 3 | 632.99 | 923.53 | y8 |
| | | | 3 | 632.99 | 1060.59 | y9 |
| | | | 3 | 632.99 | 1131.63 | y10 |
| SEQ ID NO: 3 | FEVENLILHK | 1241.68885 | 2 | 621.35 | 397.26 | y3 |
| | | | 2 | 621.35 | 510.34 | y4 |
| | | | 2 | 621.35 | 623.42 | y5 |
| | | | 2 | 621.35 | 737.47 | y6 |
| | | | 2 | 621.35 | 866.51 | y7 |
| | | | 2 | 621.35 | 965.58 | y8 |
| | | | 2 | 621.35 | 1094.62 | y9 |
| | | | 2 | 621.35 | 1241.69 | y10 |
| | | | 3 | 414.57 | 510.34 | y4 |
| | | | 3 | 414.57 | 623.42 | y5 |
| | | | 3 | 414.57 | 737.47 | y6 |
| | | | 3 | 414.57 | 866.51 | y7 |
| | | | 3 | 414.57 | 965.58 | y8 |
| | | | 3 | 414.57 | 1094.62 | y9 |
| SEQ ID NO: 4 | []GGEFTTIENQPWFAAIYR | 2326.18664 | 2 | 1163.60 | 451.27 | y3 |
| | | | 2 | 1163.60 | 522.30 | y4 |
| | | | 2 | 1163.60 | 593.34 | y5 |
| | | | 2 | 1163.60 | 740.41 | y6 |
| | | | 2 | 1163.60 | 926.49 | y7 |
| | | | 2 | 1163.60 | 1023.54 | y8 |
| | | | 2 | 1163.60 | 1151.60 | y9 |
| | | | 2 | 1163.60 | 1265.64 | y10 |
| | | | 2 | 1163.60 | 1394.69 | y11 |
| SEQ ID NO: 5 | KEDYIVYLGR | 1255.66811 | 2 | 628.34 | 720.44 | y6 |
| | | | 2 | 628.34 | 883.50 | y7 |
| | | | 2 | 628.34 | 998.53 | y8 |
| | | | 2 | 628.34 | 1127.57 | y9 |
| | | | 2 | 556.30 | 389.24 | y3 |
| | | | 2 | 556.30 | 518.28 | y4 |
| | | | 2 | 556.30 | 615.33 | y5 |
| | | | 2 | 556.30 | 712.39 | y6 |
| | | | 2 | 556.30 | 799.42 | y7 |
| | | | 2 | 556.30 | 886.45 | y8 |
| | | | 2 | 556.30 | 983.50 | y9 |
| | | | 2 | 556.30 | 1111.60 | y10 |
| SEQ ID NO: 6 | MTLTGIVSWGR | 1220.6456 | 2 | 610.83 | 418.22 | y3 |
| | | | 2 | 610.83 | 505.25 | y4 |
| | | | 2 | 610.83 | 604.32 | y5 |
| | | | 2 | 610.83 | 717.40 | y6 |
| | | | 2 | 610.83 | 774.43 | y7 |
| | | | 2 | 610.83 | 875.47 | y8 |
| | | | 2 | 610.83 | 988.56 | y9 |
| | | | 2 | 610.83 | 1089.61 | y10 |
| | | | 2 | 610.83 | 1220.65 | y11 |
| SEQ ID NO: 7 | SDALQLGLGK | 1001.56259 | 2 | 501.28 | 317.22 | y3 |
| | | | 2 | 501.28 | 374.24 | y4 |
| | | | 2 | 501.28 | 487.32 | y5 |
| | | | 2 | 501.28 | 615.38 | y6 |
| | | | 2 | 501.28 | 728.47 | y7 |
| | | | 2 | 501.28 | 799.50 | y8 |
| | | | 2 | 501.28 | 914.53 | y9 |
| | | | 2 | 501.28 | 1001.56 | y10 |
| SEQ ID NO: 8 | VSHFLPWIR | 1154.64693 | 2 | 577.83 | 474.28 | y3 |
| | | | 2 | 577.83 | 571.33 | y4 |
| | | | 2 | 577.83 | 684.42 | y5 |
| | | | 2 | 577.83 | 831.49 | y6 |
| | | | 2 | 577.83 | 968.55 | y7 |
| | | | 2 | 577.83 | 1055.58 | y8 |
| | | | 2 | 577.83 | 1154.65 | y9 |
| | | | 3 | 385.55 | 474.28 | y3 |
| | | | 3 | 385.55 | 571.33 | y4 |
| | | | 3 | 385.55 | 684.42 | y5 |
| | | | 3 | 385.55 | 831.49 | y6 |
| | | | 3 | 385.55 | 968.55 | y7 |

TABLE 2
PAI-1 Peptide Sequences and Specific Characteristics

| | | Mono- | Precursor | | Product | |
|---|---|---|---|---|---|---|
| | | isotopic | charge | Precursor | Transition | Ion |
| SEQ ID | Peptide sequence | Mass | state | m/z | m/z | Type |
| SEQ ID NO: 9 | EGSAVHHPPSYVAHLASDFGVR | 2333.14215 | 3 | 778.39 | 864.46 | y8 |
| | | | 3 | 778.39 | 1001.52 | y9 |
| | | | 3 | 778.39 | 1072.55 | y10 |
| | | | 3 | 778.39 | 1171.62 | yll |
| | | | 3 | 778.39 | 1334.69 | y12 |
| | | | 3 | 778.39 | 1421.72 | y13 |
| | | | 2 | 1167.07 | 1171.62 | y11 |
| | | | 2 | 1167.07 | 1334.69 | y12 |
| | | | 2 | 1167.07 | 1421.72 | y13 |
| SEQ ID NO: 10 | FSLETEVDLR | 1208.61574 | 2 | 604.81 | 631.34 | y5 |
| | | | 2 | 604.81 | 732.39 | y6 |
| | | | 2 | 604.81 | 861.43 | y7 |
| | | | 2 | 604.81 | 974.51 | y8 |
| SEQ ID NO: 11 | GMISNLLGK | 932.52336 | 2 | 466.77 | 544.34 | y5 |
| | | | 2 | 466.77 | 631.38 | y6 |
| | | | 2 | 466.77 | 744.46 | y7 |
| SEQ ID NO: 12 | KPLENLGMTDMFR | 1551.7658 | 2 | 776.39 | 800.34 | y6 |
| | | | 2 | 776.39 | 857.36 | y7 |
| | | | 2 | 776.39 | 970.45 | y8 |
| | | | 2 | 776.39 | 1084.49 | y9 |
| | | | 2 | 776.39 | 1213.53 | y10 |
| | | | 2 | 776.39 | 1326.62 | yl1 |
| SEQ ID NO: 13 | LVQGFMPHFFR | 1378.70887 | 2 | 689.86 | 703.37 | y5 |
| | | | 2 | 689.86 | 834.41 | y6 |
| | | | 2 | 689.86 | 981.48 | Y7 |
| | | | 2 | 689.86 | 1038.50 | y8 |
| | | | 2 | 689.86 | 1166.56 | y9 |
| SEQ ID NO: 14 | QVDFSEVER | 1108.52693 | 2 | 554.77 | 619.30 | y5 |
| | | | 2 | 554.77 | 766.37 | y6 |
| | | | 2 | 554.77 | 881.40 | y7 |
| SEQ ID NO: 15 | TPFPDSSTHR | 1144.53816 | 2 | 572.77 | 587.29 | y5 |
| | | | 2 | 572.77 | 702.32 | y6 |
| | | | 2 | 572.77 | 799.37 | y7 |
| | | | 2 | 572.77 | 946.44 | y8 |
| | | | 2 | 572.77 | 1043.49 | y9 |
| | | | 2 | 572.77 | 1144.54 | y10 |
| SEQ ID NO: 16 | VFQQVAQASK | 1105.60003 | 2 | 553.30 | 603.35 | y6 |
| | | | 2 | 553.30 | 731.40 | y7 |
| | | | 2 | 553.30 | 859.46 | y8 |
| | | | 2 | 553.30 | 1006.53 | y9 |
| | | | 2 | 553.30 | 1105.60 | y10 |
| | | | 2 | 688.87 | 704.37 | y6 |
| | | | 2 | 688.87 | 775.41 | y7 |
| | | | 2 | 688.87 | 874.47 | y8 |
| | | | 2 | 688.87 | 1002.53 | y9 |
| | | | 2 | 688.87 | 1130.59 | y10 |
| SEQ ID NO: 17 | VHHPPSYVAIILASDFGVR | 1989.00895 | 4 | 498.01 | 593.30 | y5 |
| | | | 4 | 498.01 | 680.34 | y6 |
| | | | 4 | 498.01 | 751.37 | y7 |
| | | | 4 | 498.01 | 864.46 | y8 |
| | | | 4 | 498.01 | 1001.52 | y9 |
| | | | 4 | 498.01 | 1072.55 | y10 |
| | | | 4 | 498.01 | 1171.62 | y11 |
| | | | 4 | 498.01 | 1334.69 | y12 |
| | | | 4 | 498.01 | 1421.72 | y13 |
| | | | 2 | 995.01 | 1001.52 | y9 |
| | | | 2 | 995.01 | 1072.55 | y10 |
| | | | 2 | 995.01 | 1171.62 | y11 |
| | | | 2 | 995.01 | 1334.69 | y12 |
| | | | 2 | 995.01 | 1421.72 | y13 |
| SEQ ID NO: 18 | VFQQVAQASKDR | 1376.72809 | 2 | 688.87 | 704.37 | y6 |
| | | | 2 | 693.87 | 714.38 | y6 |
| | | | 2 | 688.87 | 775.41 | Y7 |
| | | | 2 | 693.87 | 785.41 | y7 |
| | | | 2 | 688.87 | 874.47 | y8 |
| | | | 2 | 693.87 | 884.48 | Y8 |
| | | | 2 | 688.87 | 1002.53 | y9 |
| | | | 2 | 693.87 | 1012.54 | y9 |
| | | | 2 | 688.87 | 1130.59 | y10 |

1. A method for measuring levels of the uPA and PAI-1 proteins in a biological sample, comprising detecting at least one fragment peptide from uPA and at least one fragment peptide from PAI-1 in a protein digest prepared from said biological sample using mass spectrometry; and calculating the levels of the uPA and PAI-1 proteins in said sample, wherein said measured levels of the uPA and PAI-1 proteins are independently selected from a relative level or an absolute quantitative level.

2. The method of claim 1, further comprising the step of fractionating said protein digest prior to detecting said peptides.

3-4. (canceled)

5. The method of claim 1, wherein said protein digest comprises a protease digest.

6-9. (canceled)

10. The method of claim 1, wherein the uPA fragment peptide comprises an amino acid sequence as shown in Table 1, and set forth as SEQ ID NO:1, SEQ ID NO:2, SEQ ID NO:3, SEQ ID NO:4, SEQ ID NO:5, SEQ ID NO:6, SEQ ID NO:7, and SEQ ID NO:8.

11. (canceled)

12. The method of claim 1, wherein the PAI-1 fragment peptide, or peptides, comprises an amino acid sequence as shown in Table 2, and set forth as SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, and SEQ ID NO:18.

13. The method of claim 12, wherein uPA peptide identification and characteristics are defined as shown in Table 2 by its specified monoisotopic mass, specified precursor charge state, specified precursor mass over charge ratio (m/z), specified product transition ions (m/z), and specified ion type.

14. The method of claim 1, wherein the biological sample comprises blood, urine, serum, ascites, saliva, cells, or tissue.

15. The method of claim 14, wherein the tissue is formalin fixed tissue.

16. (canceled)

17. The method of claim 14, wherein the tissue is obtained from a tumor.

18.-19. (canceled)

20. The method of claim 1, further comprising quantifying the uPA fragment peptide, or peptides.

21-22. (canceled)

23. The method of claim 1, further comprising quantifying the PAI-1 fragment peptide, or peptides.

24-27. (canceled)

28. The method of claim 1, further comprising obtaining the biological sample from a subject, wherein detecting the uPA and PAI-1 fragment peptides in the protein digest indicates the presence of uPA and PAI-1 and an association with cancer in the subject.

29. The method of claim 28, further comprising correlating detected and quantitated amounts of the uPA and PAI-1 fragment peptides to the diagnostic stage/grade/status of the cancer.

30. (canceled)

31. The method of any one of claim 20 or 23, further comprising selecting a therapeutic treatment for the subject based on the presence, absence, or quantified levels of the uPA and PAI-1 fragment peptides in the protein digest.

32. The method of any one of claim 20 or 23, further comprising administering a therapeutically effective amount of a therapeutic agent targeted specifically to the uPA and/or PAI-1 proteins or the level of uPA and/or PAI-1 proteins, wherein the treatment decision about which agent or agents, or the amount of said agent, or agents, used for treatment is based upon specific levels of the uPA and/or PAI-1 fragment peptides in the biological sample.

33. The method of claim 32, wherein therapeutic agents include those that specifically bind to uPA and/or PAI-1 proteins and inhibit the biological activity of either uPA or PAI-1.

34-36. (canceled)


SEQUENCE LISTING (18 sequences)

SEQ ID NO: 1 - protein, 10 aa, Artificial Sequence
  uPA proteolytic peptide
KPSSPPEELK

SEQ ID NO: 2 - protein, 17 aa, Artificial Sequence
  uPA proteolytic peptide
DYSADTLAHHNDIALLK

SEQ ID NO: 3 - protein, 9 aa, Artificial Sequence
  uPA proteolytic peptide
FEVENLILH

SEQ ID NO: 4 - protein, 20 aa, Artificial Sequence
  uPA proteolytic peptide
IIGGEFTTIENQPWFAAIYR

SEQ ID NO: 5 - protein, 10 aa, Artificial Sequence
  uPA proteolytic peptide
KEDYIVYLGR

SEQ ID NO: 6 - protein, 11 aa, Artificial Sequence
  uPA proteolytic peptide
MTLTGIVSWGR

SEQ ID NO: 7 - protein, 10 aa, Artificial Sequence
  uPA proteolytic peptide
SDALQLGLGK

SEQ ID NO: 8 - protein, 9 aa, Artificial Sequence
  uPA proteolytic peptide
VSHFLPWIR

SEQ ID NO: 9 - protein, 22 aa, Artificial Sequence
  PAI-1 proteolytic peptide
EGSAVHHPPSYVAHLASDFGVR

SEQ ID NO: 10 - protein, 10 aa, Artificial Sequence
  PAI-1 proteolytic peptide
FSLETEVDLR

SEQ ID NO: 11 - protein, 9 aa, Artificial Sequence
  PAI-1 proteolytic peptide
GMISNLLGK

SEQ ID NO: 12 - protein, 13 aa, Artificial Sequence
  PAI-1 proteolytic peptide
KPLENLGMTDMFR

SEQ ID NO: 13 - protein, 11 aa, Artificial Sequence
  PAI-1 proteolytic peptide
LVQGFMPHFFR

SEQ ID NO: 14 - protein, 9 aa, Artificial Sequence
  PAI-1 proteolytic peptide
QVDFSEVER

SEQ ID NO: 15 - protein, 10 aa, Artificial Sequence
  PAI-1 proteolytic peptide
TPFPDSSTHR

SEQ ID NO: 16 - protein, 10 aa, Artificial Sequence
  PAI-1 proteolytic peptide
VFQQVAQASK

SEQ ID NO: 17 - protein, 18 aa, Artificial Sequence
  PAI-1 proteolytic peptide
VHHPPSYVAHLASDFGVR

SEQ ID NO: 18 - protein, 12 aa, Artificial Sequence
  PAI-1 proteolytic peptide
VFQQVAQASKDR